(19) United States
(12) Patent Application Publication
(10) Pub. No.: US 2004/0068263 A1
(43) Pub. Date: Apr. 8, 2004

(54) CAS bone reference with articulated support

(76) Inventors: Benoit Chouinard, Montreal (CA); Herbert Jansen, Montreal (CA); Sebastien Jutras, Montreal (CA); Sebastien Cossette, Montreal (CA)

Correspondence Address: OGILVY RENAULT, 1981 MCGILL COLLEGE AVENUE, SUITE 1600, MONTREAL, QC, H3A2Y3, CA

(21) Appl. No.: 10/263,711

(22) Filed: Oct. 4, 2002

(51) Int. Cl. A61F 5/00

(52) U.S. Cl. 606/86

(57) ABSTRACT

A surgical bone reference assembly adapted for communication with an image guided surgical system. A bone anchor member is engageable to a bone element of a patient such that substantially no relative movement therebetween is possible. A trackable member comprises a detectable element adapted to be located and tracked in three dimensional space by the image guided surgical system, thereby defining position and movement of the trackable member. An adjustable articulated support member links the trackable member and the bone anchor member, and permits variable positioning of the trackable member relative to the bone anchor member, while being lockable to fix the trackable member in position relative to the bone anchor member.

This application incorporates by reference concurrently filed application titled “CAS Bone Reference And Less Invasive Installation Method Thereof”, commonly assigned to ORTHOsoft Inc. by Sébastien Cossette et al. and having attorney docket number 15228-23us.

### TECHNICAL FIELD

The present invention relates generally to a trackable reference for use in conjunction with a Computer Assisted Surgery (CAS) system. More particularly, the present invention relates to a bone reference having a selectively articulated support for a position identifying element trackable by the CAS system.

### BACKGROUND OF THE INVENTION

CAS systems capable of real time location and tracking of a plurality of discrete objects in a surgical field are now becoming increasingly well known. A variety of systems are employed, however all require the patient bone elements to be identified and registered to pre-operatively taken anatomical scans or intra-operatively taken images of the same bone elements. In order for the relevant bone elements to be located and tracked by the CAS system, trackable reference members must be fastened thereto. These bone reference members will vary depending on the type and specific requirements of the particular CAS system used.

For example, for an optical CAS system, the trackable bone reference members will comprise at least three optically detectable markers whose exact positions can be determined by each of the at least two cameras of the optical CAS system. This therefore permits the position in space of each detectable marker to be determined by the CAS system, and therefore permits the position and orientation of the bone reference member, and consequently also the position and orientation of the bone element to which it is affixed, to be determinable by the CAS system.

No matter what type of positioning reference block is used, all such reference members used in conjunction with a computer assisted surgery must comprise a trackable member. It is well known to permanently fix such trackable members to the reference block by such methods as welding, press-fitting, and pinning. However, as it can be desirable in particular circumstances to be able to separate the trackable member portion from the base reference block, it is known to fasten the trackable member to the reference block with releasable engagement mechanisms. These generally permit the trackable member to be completely removed from the reference member fastened to the bone element. This can be useful, for example, if temporary removal of the trackable member provides better access for the surgeon to a particular location. Additionally, quick-release removal of the trackable member and replacement in an alternate position on the reference block is known, and can be useful to ensure the best line of sight between the detectable element of the trackable member and the cameras of the CAS system, for applications where the same reference instrument can be employed in selected different anatomical locations. For example, a bone reference member used in knee surgery must be equally practical when used on both the right and left knees, however to ensure the optimal position of the trackable member within surgical field of the CAS system cameras, it is generally desirable to be able to switch the trackable member from one side of the reference block to the other.

However, once the trackable member is removed from the reference block fixed to the bone element, the position and orientation of the bone element is no longer known. As such, when the trackable member is reattached to the reference member in an alternate position, the bone element must be re-registered in order for the CAS model or image to correspond to the position and orientation of the actual bone element, and such that the reference member can then be again used to accurately track the bone element to which it is fixed.

Additionally, for CAS systems such as those that are optically based, the ability to maintain an unobstructed line of sight view between the system cameras and the detectable marker elements of the trackable member is of prime importance. This can, however, become difficult in some surgical installations, where numerous medical staff and a large quantity of medical equipment are required within the surgical field. The cameras of the CAS system must be able to simultaneously visually locate both the bone reference trackable member and any additional trackable members disposed on tracked tools employed. While tracked surgical instruments can more easily be displaced such that their trackable members are in an optimal position relative to the cameras, it is often more difficult and impractical to adjust the trackable bone reference member, being fastened to a bone element of the patient.

Therefore, while the ability to remove or displace a trackable member relative to a bone reference block to which it is engageable is desirable, completely removing the trackable member and re-installing it in an alternate position is time consuming and complex. The re-registration that is then subsequently required is also further time consuming, resulting in the removal or adjustment of the trackable member relative to the fixed bone reference block being substantially impractical intra-operatively. Additionally, known bone reference members provide limited tracker adjustability and consequently maintaining an optimal, unobstructed visual contact between the bone reference trackable member and the cameras of the CAS system is often difficult.

### SUMMARY OF THE INVENTION

It is accordingly an object of the present invention to provide an improved CAS bone reference assembly having a trackable member adapted for communication with an image guided surgical system.

It is another object of the present invention to provide a CAS bone reference assembly having a trackable member that is selectively adjustable relative to a base reference member to which it is engaged.

It is another object of the present invention to provide a CAS bone reference assembly comprising an articulated support for a trackable member permitting at least two degrees of freedom relative to a base reference member to which it is engaged.

Therefore, in accordance with the present invention, there is provided a surgical bone reference assembly, adapted for communication with an image guided surgical system, comprising: a bone anchor member, engageable to a bone element of a patient such that substantially no relative movement therebetween is possible; a trackable member comprising a detectable element adapted to be located and tracked in three dimensional space by the image guided surgical system, thereby defining position and movement of the trackable member; and an adjustable articulated support member linking the trackable member and the bone anchor member, the adjustable articulated support member permitting variable positioning of the trackable member relative to the bone anchor member and being lockable to fix the trackable member in position relative to the bone anchor member.

There is also provided, in accordance with the present invention a method for monitoring position and movement of a bone element using an image guided surgical system, comprising the steps of: adjusting a trackable member of a bone reference assembly into a desired position and orientation relative to sensing elements of the image guided surgical system, the trackable member having detectable elements being locatable and trackable in three dimensional space by the image guided surgical system; locking an adjustable articulated support member such that the trackable member is fixed in the desired position, the adjustable articulated support member linking the trackable member to a bone anchor member adapted to be fastened to the bone element; performing a registration of the bone element; and locating and tracking the bone element using the image guided surgical system.

There is further provided, in accordance with the present invention, a computer assisted surgical system capable of locating and tracking a bone element in three dimensional space, comprising: a bone reference assembly having a trackable member being communicable with at least a sensing element of the system; an anchor member fastenable to the bone element; an articulated support member linking the trackable member to the anchor member; and means for determining a preferred position of the trackable member relative to the sensing element of the system.

### BRIEF DESCRIPTION OF THE DRAWINGS

Further features and advantages of the present invention will become apparent from the following detailed description, taken in combination with the appended drawings, in which:

FIG. 1 is a front perspective view of a surgical bone reference assembly according to the present invention.

FIG. 2 is a front elevation view of the surgical bone reference assembly of FIG. 1, but shown engaged to a bone element of a patient.

FIG. 3 is a side elevation view of the surgical bone reference assembly of FIG. 1.

FIG. 4 is a rear perspective view of the surgical bone reference assembly of FIG. 1.

### DETAILED DESCRIPTION OF THE PREFERRED EMBODIMENT

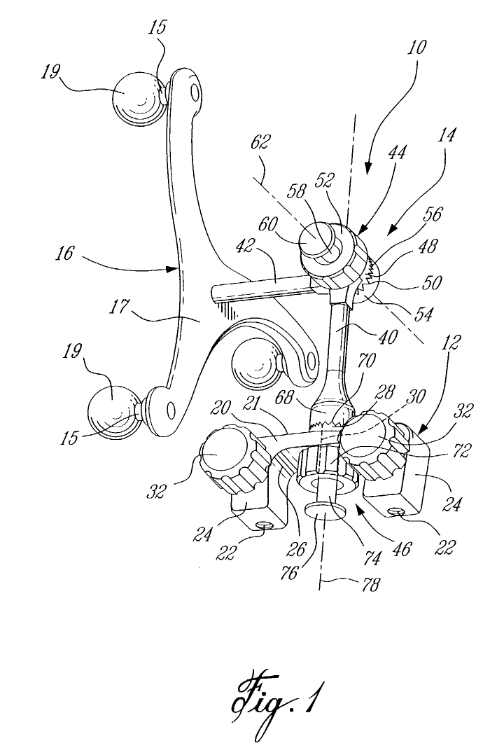

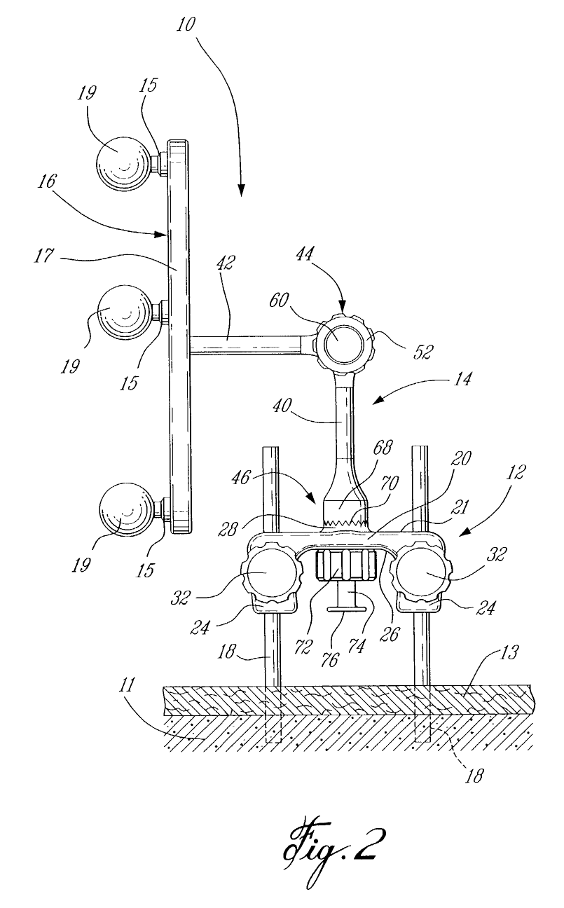

Referring to FIG. 1, the surgical bone reference assembly 10 comprises generally a bone anchor member 12, an articulated tracker support 14 and a trackable member 16, preferably adapted to be communicable with an image guided, computer assisted surgery (CAS) system capable of detecting and tracking the device in three-dimensional space within surgical field. The bone anchor block 12 comprises a central bridge portion 20, linking two proximally extending and integrally formed legs portions 24. A central opening 26, defined on three sides by the opposing lateral leg portions 24 and the distal central bridge portion 20, opens towards the patient to which the surgical bone reference assembly 10 is to be engaged. At least two positioning pin holes 22 are defined within the leg portions 24. As best seen in FIG. 2, the pin holes 22 extend through the leg portions 24 to permit the bone anchor block 12 to be fixed in place on positioning pins 18 which are fastened into a bone element 11 of a patient, such that no movement of the anchor member 12 relative to the bone element is possible.

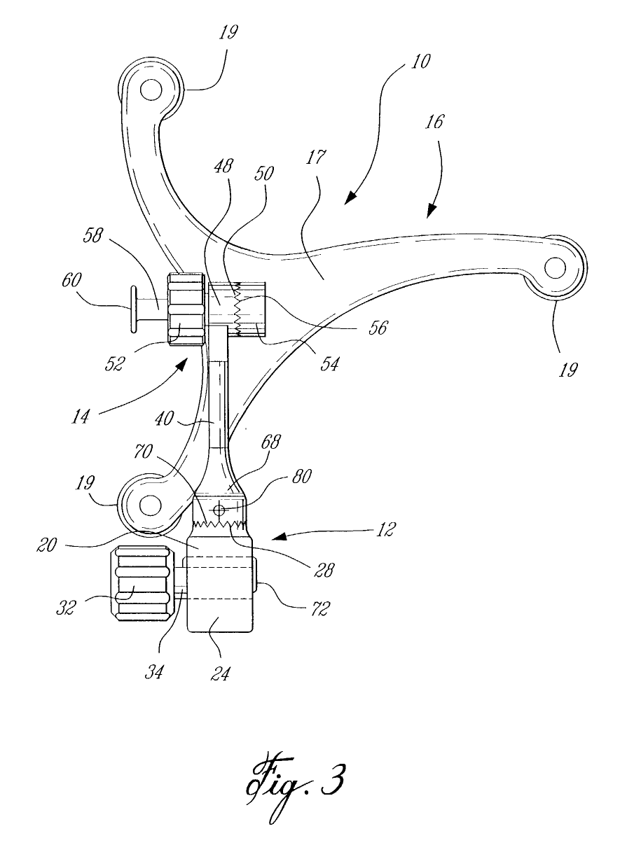

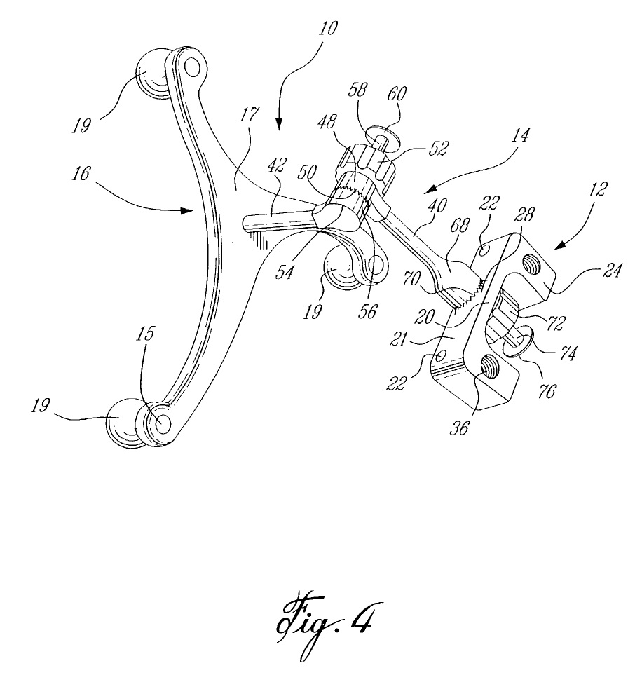

Bone pin locking screws 32 are preferably used to fasten the anchor block 12 to the positioning pins 18. As best seen in FIGS. 3 and 4, the locking screws 32 comprise threaded bodies 34 which engage tapped holes 36, perpendicularly disposed relative to, and intersecting, the pin holes 22. This therefore permits the tips of the locking screws 32 to frictionally engage the locating pins 18, such that the anchor member 12 can be fixed in place thereon. Consequently, the bone anchor member 12 can be fixed relative to the patient, without being directly fastened thereto. While the pin holes 22 as shown in the figures are parallel to one another, the pin holes 22 can alternately have a slight inclination angle, such that they are inclined proximally inwards. This requires each of the positioning pins 18 to be anchored into the bone element 11 at a corresponding angle. With the positioning pins 18 extending distally away from one another, better stability is provided for the anchor member 12 when engaged thereto. When the pin holes 22 are aligned with the positioning pins 18 and the anchor member 12 is pressed downward toward the patient, the divergently inclined positioning pins 18 limit the movement of the anchor member 12 thereon, thereby improving bone anchoring stability and further providing substantially fixed engagement between the positioning pins 18 and the anchor member 12. Preferably, the pins are inclined about 6.5 degrees away from an axis perpendicular to the bone surface, and consequently the pin holes 22 are correspondingly angled at about 6.5 degrees from an axis perpendicular to the distal surface 21 of the anchor member 12. While an angle of 6.5 degrees is best, any substantially smaller or larger angle can also be used. Generally, using a larger inclination angle may require a bigger anchor member, and using a smaller inclination angle will permit the anchor member to slide much further down the positioning pins and may perhaps contact the body member to which the positioning pins are fastened.

Only the limitedly invasive positioning pins 18, to which the bone anchor member 12 is preferably engaged above the surface of the soft tissue 13 and skin, are directly fastened in the bone element 11. This permits relatively reduced installation invasiveness, as the pins 18 can be directed through small incisions in the soft tissue 13. Although at least two traditional bone anchored positioning pins 18 are preferably used to fasten the present bone reference assembly 10 to a bone element 11, it is also possible to engage the bone anchor member 12 to at least one, non-circular positioning pin or rod. A positioning rod having a non-circular cross-sectional area received into a correspondingly shaped aperture or bore in the bone anchor member 12, would similarly prevent the possibility of the reference assembly 10 from rotating relative to the bone element 11, and the anchor member 12 could similarly be axially fastened thereto. This alternate installation would equivalently eliminate any relative movement between the anchor member 12 and the bone element 11, while requiring only a single insertion point for mounting the bone reference assembly 10 to the bone element 11, thereby further reducing installation invasiveness. Two pins with such non-circular cross-sectional area are similarly feasible.

The trackable member 16 generally comprises a detectable tracker head element 17, including detectable element mounting posts 15 for receiving detectable markers 19 thereon, that is connected to the bone anchor member 12 by an articulated support member 14 that will be described in further detail below. To each mounting post 15 is removably fixed a detectable marker element, such as an optically detectable sphere element 19. The detectable spheres 19 are preferably coated with a retro-reflective layer in order to be detected by, for example, an infrared sensor using axial illumination. Cameras of the optical CAS system can therefore detect the position of each optically detectable sphere 19 illuminated by infrared light. Each detectable marker element 19 can equally be any other type of position indicator such as a light emitting diode or detectable electromagnetic indicator, provided each can be detected by the type of sensor used by the CAS system. Although the present surgical bone reference assembly 10 is preferably adapted for use with an optically based CAS system, one skilled in the art will appreciate that in addition to the optical and electromagnetic systems mentioned above, other types of CAS systems can also be used, such as, for example, those which use ultrasound or laser as a means for position identification. In such cases, it is to be understood that the detectable sphere elements 19 will be such that they are able to indicate to, or be detected by, the particular CAS position identification system used.

The articulated support 14 preferably permanently links the trackable member 16 to the anchor member 12, such that the trackable member 16 cannot be completely separated from the base bone anchor member 12. The articulated support 14 also permits selective adjustability of the position in space of the trackable member 16 relative to the bone anchor member 12 that is fixed to the bone element 11. The articulated support member 14 preferably comprises at least two independently articulated joints. However, a single joint is equally envisionable. For example, a single rotating joint can be used between the bone anchor member 12 and an angled, rigid support arm having a trackable member on the end thereof. Although providing less adjustability and range of motion, such an arrangement would be simpler and less expensive. No matter the number, each joint preferably provides an independent single degree of freedom. However, a selectively lockable, ball-and-socket type joint could also be used, and would provide itself three rotational degrees of freedom. While joints providing rotational movement are preferred, other types of joints, for example those providing a translational degree of freedom, are equally possible, but preferably used in combination with at least one rotational joint.

Referring to the preferred embodiment as depicted in FIGS. 1 to 4, the articulated support member 14 comprises a first link member 40 and a second link member 42, interconnected by a first joint assembly 44 therebetween. The second link member 42 comprises a rigid rod element, fixed at one end to the tracker head element 17 of the trackable member 16, and having a preferably integrally formed annular second link end 54 at an opposing end. The annular second link end 54 includes a serrated, or toothed ring 56, disposed substantially perpendicularly to the surface of the tracker head element 17. The toothed ring 56 is preferably integrally formed with the annular second link end 54. The serrations or teeth of the toothed ring 56 inter-engage with corresponding teeth of a toothed ring 50, preferably integrally formed on an annular first link end 48 of the first link member 40. When the two toothed rings 50 and 56 are pressed into engagement together, the teeth interlock to prevent rotational movement relative to one another. The annular first link end 48 comprises a central aperture defined therethrough, about which the toothed ring 50 disposed. The central aperture in the distal first link end 48 is concentric with the first joint axis of rotation 62, substantially perpendicular to a longitudinal axis of the first link member 40. A first axle pin 58 is permanently fixed at one end to the second link end 54, and extends through the central aperture in the annular first link end 48. The first joint axle pin 58 has an externally threaded central portion, not seen in the figures but disposed generally partially beneath each of a first joint locking nut 52 and the annular first link end 48. The central aperture through the first link end 48 has a diameter sufficiently large enough such that the axle pin 58 is free to rotate within the aperture. The axle pin 58 also comprises a disc flange 60 at the free end of the pin 58 opposed to the end fixed to the second link end 54. The disc flange 60 prevents the first joint locking nut 52 from being completely separable from the first joint assembly 44. When the locking nut 52, having internal threads corresponding to those on the axle pin 58, is tightened, it forces the annular first link end 48 towards the second link end 54, such that the corresponding toothed rings 50 and 56 engage one another. This thereby engages the first and second link members 40 and 42 in a specific angular relation to one another. The first joint assembly 44 therefore permits selective rotational adjustment of the second link member 42, to which the trackable member 16 is fastened, about the first axis of rotation 62.

The articulated support 14 further comprises a second joint assembly 46, providing selective rotational adjustment between the first link member 40 and the bone anchor member 12 about the second joint axis of rotation 78, collinear with the longitudinal axis of the first link member 40. The second joint assembly 46 operates much as the first joint assembly 44, permitting selective rotation of the first link member 40 relative to the bone anchor member 12 when a second joint locking nut 72 is disengaged, and fixed engagement between the anchor member 12 and the first link member 40 when the second joint locking nut 72 is tightened. The second joint assembly 46 includes a proximal first link end 68, disposed at an opposite end of the first link member 40 from the distal first link end 48. The proximal first link end 68 comprises a toothed ring 70, having proximally projecting teeth for engagement with the distally projecting teeth of a corresponding toothed ring 28, centrally disposed on a distal surface 21 of the bridge portion 20 of the bone anchor member 12. Within the toothed ring 28 is a concentric central circular aperture, bored through the bridge portion 20 of the bone anchor member 12, and through which extends a second joint axle pin 74. A distal end of the second joint axle pin 74 is permanently fastened to the proximal end 68 of first link member 40, in an aperture concentric with the toothed ring 70. Much as the first joint axle pin 58, the second joint axle pin 74 has a threaded central body portion, such that the second joint locking nut 72 can be engaged thereto, thereby forcing the first link member 40 into fixed engagement with the bone anchor member 12 when the locking nut 72 is tightened. The corresponding teeth of the mating toothed rings 70 and 28 on both the proximal end 68 of the first link member 40 and the distal surface 21 of the bone anchor member 12, are consequently engaged such that these two components are rotationally fixed relative to one another.

Although any method can be used to permanently fasten the axle pin 74 within the proximal end of the first link member 40, a small cross-pin fastener is preferably used. As best seen in FIG. 3, a small pin 80 transversely extends through the first link member proximal end 68 and through the axial second joint axle pin 74 to thereby permanently fasten them together. It is to be noted that, when fastening the joint axle pins 58 and 74 of both joint assemblies 44 and 46 to their link member ends, the axle pins must first be threaded onto the locking nuts 52 and 72, as the free end flanges 60 and 76 of the joint axle pins 58 and 74 would prevent the locking nuts from being installed once the axle pins are fastened in place. This enables the locking nuts 52 and 72, although they can be fully disengaged from the threads of the joint axle pins 58 and 74, to be permanently captive such that they can never inadvertently fall off if completely unscrewed.

An important feature of the entire present bone reference assembly 10, is the ease with which all surfaces of the assembly can be cleaned. Particularly, all surfaces of the joints can be sufficiently exposed such that thorough pressure cleaning is possible. The ability to pressure clean and autoclave all surfaces of surgical devices is vital to ensure that all contaminating biological matter can be safely removed. Such potentially dangerous contaminating biological matter can include unwanted bacteria and prions, microscopic protein particles similar to those of a virus but lacking nucleic acid and thought to be an infectious agent responsible for certain degenerative diseases of the nervous system. The free end flanges 60 and 76 of the joint axle pins 58 and 74 are spaced sufficiently away from the joint that the joint locking nuts 52 and 72 can be completely unscrewed and the two halves of the joints separated such that all surfaces, including the outer threads of the joint axle pins, can be substantially exposed to permit pressure cleaning thereof.

Construction of the surgical bone reference assembly 10 is preferably made such that the trackable member 16 is permanently linked to the base bone anchor member 12, but can be selectively adjusted in order to ensure the best line-of-sight communication between the detectable sphere elements 19 and the cameras of the CAS system. Once the bone anchor member 12 is engaged to the bone-embedded positioning pins 18 as described above, and with the two joint locking nuts 52 and 72 being loosened sufficiently to permit free movement of the two link members of the articulated support 14, the trackable member 16 can be moved by the surgeon until the trackable member is in a suitable position. This can constitute a position that best permits unobstructed access of the patient for the surgical procedure, and/or a position that best permits clear communication between the cameras or other similar position sensing elements of the CAS system and the detectable sphere elements 19 of the trackable member 16. Such a desired most suited position for the trackable member 16 can be either chosen by the surgeon without any guidance, or the CAS system can provide visual or audible indication to the surgeon when the trackable member 16 has reached a position of clear communication with the cameras, for example. Either way, once the trackable member 16 is in the desired final position, the joint locking nuts 52 and 72 of the articulated support assembly 14 are tightened, thereby engaging the trackable member 16 fixed relative to the bone anchor member 12 and consequently the bone element 11 of the patient. Other joint locking methods can equivalently be used in place of the screwed joint locking nuts 52 and 72. Any mechanism that similarly permits two adjacent link members to be temporarily fixed together at a joint therebetween can equivalently replace the locking nuts 52 and 72. For example, if the joint axle pins can comprise keyways or teeth corresponding to, and normally engaged with, similar element on the link ends. The joint axle pins are normally biased such that they are in meshed engagement with the two link ends and retain them together, but can be slid outward and out of engagement with the two link member ends, such that the joint is unlocked while the joint axle pin is held disconnected, thereby permitting free movement of the two link member relative to one another. Similarly, the joints can comprise a lockable ratcheting mechanism, such that rotation between link members interconnected by such a joint is normally prevented. The mechanism comprises a biased pawl on one link member end which engages a toothed wheel disposed on the adjacent link member end, such that only when the biased pawl is selectively disengaged from the opposing wheel will the joint permit free rotational relative movement between the two link members.

In an alternate embodiment not depicted, the joints of the articulated trackable member support comprise graduations or markings about the circumference of both elements of each rotational joint. For example, the first joint 44 has regularly spaced graduations on the circumferential surfaces of both the annular first link end 48 and the second link end 54, just adjacent the toothed rings 50 and 56. This permits the position in which the articulated support assembly 14 is fixed to be quantifiably identified, and, should the joints be disengaged and the trackable member 16 be displaced to another location, this would then enable the articulated support to be moved back to the original configuration by re-aligning the necessary joint markings. This permits, for example, the trackable member 16 to be temporarily displaced intra-operatively to improve access to the patient with a particular surgical instrument and subsequently accurately returned to the original position, without necessitating a new registration of the trackable member 16 relative to the bone element 11. This represents a significant time savings, compared to traditional bone reference blocks which, once displaced, had to be completely re-registered with the CAS system before continuing.

In another alternate embodiment, the CAS system prompts the surgeon for the initial angles between the links of the articulated support 14 as fixed in place. In order to alter the position of the trackable member 16 intra-operatively, the surgeon advises the CAS system that the relative positions of the link member of the articulated support 14 are being changed and provides the system with the new angles between each link member, as visually read off the graduations on the joints of the articulated support member 14. Alternately, the CAS system itself detects when a drastic displacement of the trackable member 16 occurs, and prompts the user to verify if the trackable member has be moved relative to the bone element 11. If it is told that this in fact has occurred, the system prompts the user for either the new angles or the change in angles between each link member of the articulated support 14. The CAS system can alternately recognise the new position of the trackable member 16, and calculate the position differences between the new position relative to the previous position, fixed relative to the bone anchor member 12, and calculate the displacement values accordingly. Either way, the CAS system uses the displacement values to determine a translation matrix, which is then used to re-adjust the previous position of computer model of the bone element 11 relative to the trackable member 16. By being able to re-adjust for the displacement of the detectable elements relative to their original positions, the CAS system permits the surgery to proceed without having to perform any re-registration.

As mentioned above, the CAS bone reference assembly 10 of the present invention is preferably intended to be used in conjunction with an optical tracking CAS system which employs a network of cameras to locate the trackable member 16, or more specifically to locate identification markers 19 of a detectable element 17 thereof, so that their position and movement can be tracked during the surgery. Therefore, when the bone reference assembly 10 is fixed to the desired patient bone element 11, the anatomical position and orientation of the bone element 11 can be determined and tracked in space by the CAS system. However, a registration of the bone element must first be performed. It is to be understood that the step of performing the registration of the bone element, as used herein, comprises all means of relating the actual bone element 11 to a corresponding model or image of the same bone element. Those skilled in the art will appreciate that there are a plurality of ways of creating such a model or image of the bone element, and of relating or matching the actual bone element 11 to the model or image thereof.

Generally, a plurality of points are first acquired on the surface of the bone element 11 using a CAS system communicable probe or pointer instrument. These points can then, for example, be registered to the a corresponding virtual model of the bone element 11 generated from a computed tomography (CT) scan. Similarly, the position and orientation in 3D space of reference artifacts in anatomical images of the bone element 11 can be mathematically related to the position of the reference clamp. The principle function being to permit the bone element 11 to be matched with the corresponding anatomical image or model displayed to the surgeon on a monitor, such that the real-time position of the bone element 11, to which the present bone reference assembly 10 is fixed, can be shown graphically to the surgeon. Generally, either pre-operatively taken CT scans or intra-operative fluoroscopic images of the patient are used to create the anatomical model or image which is subsequently displayed on the monitors during the surgery to provide the surgeon with an accurate representation of the specific body parts or targeted elements of the patient.

For example, when CT-based images are being used, once the bone reference assembly 10 is securely engaged to the bone element 11, thereby fixing the bone element 11 relative to the location of the trackable member 16 of the bone reference assembly 10, the bone element 11 can then be registered to the computer model element thereof. This is preferably done by acquiring a plurality of points, either pre-determined and sequentially identified by the CAS to the surgeon or randomly selected by the surgeon, on the surface of the bone element using a well known calibrated CAS probe. The points on the physical bone element are then matched with corresponding points on the 3D model, thereby registering the CAS system bone model to the tracked position in space of the anatomical counterpart.

The anatomical models or images can also be acquired and/or generated using other methods such as magnetic resonance imaging, ultrasound and/or landmark digitization techniques. Such landmark digitization techniques permit intra-operatively acquired surface points, preferably acquired on specific predetermined landmarks of the bone element surface, to be used to create a computerized anatomical reference model of the bone element. This can eliminated the need for a CT scan, taken pre-operatively for example, to be used to generate the computer reference model of the bone element. All of the above described alternate methods of generating a computerized model or displaying image of the bone element, and of relating or matching the position and orientation of the actual bone element 11 thereto, will be understood herein to be included in the process of performing a registration of the bone element.

The embodiments of the invention described above are intended to be exemplary only. The scope of the invention is therefore intended to be limited solely by the scope of the appended claims.

1. A surgical bone reference assembly, adapted for communication with an image guided surgical system, comprising:
a bone anchor member, engageable to a bone element of a patient such that substantially no relative movement therebetween is possible;
a trackable member comprising a detectable element adapted to be located and tracked in three dimensional space by the image guided surgical system, thereby defining position and movement of the trackable member; and
an adjustable articulated support member linking the trackable member and the bone anchor member, the adjustable articulated support member permitting variable positioning of the trackable member relative to the bone anchor member and being lockable to fix the trackable member in position relative to the bone anchor member.

2. The surgical bone reference assembly as defined in claim 1, wherein the articulated support member permits selective positioning of the trackable member relative to the bone anchor member between predetermined fixed positions.

3. The surgical bone reference assembly as defined in claim 2, wherein the articulated support member permits at least two degrees of freedom of the trackable member relative to the bone anchor member.

4. The surgical bone reference assembly as defined in claim 3, wherein the articulated support member comprises at least two independent joints, each enabling a respective degree of freedom.

5. The surgical bone reference assembly as defined in claim 4, wherein the independent joints comprise lockable rotational joints.

6. The surgical bone reference assembly as defined in claim 5, wherein the lockable rotational joints comprise two mating elements, each disposed on a separate link of the articulated support member, being rotatable relative to one another about a joint axis of rotation when a joint locking mechanism is disengaged, and being rotatably fixed relative to one another when the joint locking mechanism is engaged.

7. The surgical bone reference assembly as defined in claim 6, wherein the joint locking mechanism comprises corresponding toothed rings disposed on both mating elements of the lockable rotational joint concentric with the joint axis of rotation and a locking nut, the joint locking nut forcing the corresponding toothed rings together when tightened, thereby engaging the joint locking mechanism, and permitting the toothed rings to be separated when sufficiently loosened, thereby disengaging the toothed rings.

8. The surgical bone reference assembly as defined in claim 1, wherein the bone anchor member comprises at least an aperture therein, there being provided a positioning rod for each aperture, the aperture being adapted for receiving a distal end of the positioning rod, the positioning rod being adapted to be anchored at a proximal end thereof to the bone element, the bone anchor member also comprising a locking member for selective engagement with the positioning rod such that the bone anchoring member is releasably engageable thereto supra-cutaneously.

9. The surgical bone reference assembly as defined in claim 8, wherein the bone anchor member comprises at least two apertures, each adapted for receiving a bone positioning pin.

10. The surgical bone reference assembly as defined in claim 1, wherein the articulated support member permanently links the trackable member and the bone anchor member.

11. The surgical bone reference assembly as defined in claim 1, wherein all surfaces are substantially seamless and are at least one of substantially exposed and exposable, such that the surfaces can easily be pressure cleaned and autoclaved to remove biological matter therefrom.

12. A method for monitoring position and movement of a bone element using an image guided surgical system, comprising the steps of:
adjusting a trackable member of a bone reference assembly into a desired position and orientation relative to sensing elements of the image guided surgical system, the trackable member having detectable elements being locatable and trackable in three dimensional space by the image guided surgical system,;
locking an adjustable articulated support member such that the trackable member is fixed in the desired position, the adjustable articulated support member linking the trackable member to a bone anchor member adapted to be fastened to the bone element;
performing a registration of the bone element; and
locating and tracking the bone element using the image guided surgical system.

13. The method as defined in claim 12, further comprising using the image guided surgical system to determine the desired position and orientation of the trackable member, the desired position and orientation of the trackable member permitting substantially uninterrupted communication between the detectable elements of the trackable member and the sensing elements of the image guided surgical system.

14. The method as defined in claim 12, further comprising performing the registration by acquiring a number of points on a surface of the bone element, and matching the points to a corresponding surface of a computer stored representation of the bone element.

15. The method as defined in claim 14, wherein the computer stored representation is created by generating a three dimensional computer model of the bone element from a computed tomography scan.

16. The method as defined in claim 15, further comprising taking the computed tomography scan pre-operatively.

17. The method as defined in claim 12, further comprising performing the registration by matching reference artefacts from a two-dimensional fluoroscopic image of the bone element to the location of the reference artefacts at the time the fluoroscope image was taken, the position and orientation of the reference artifacts being determined by the image guided surgical system.

18. The method as defined in claim 17, further comprising taking the fluoroscopic image intraoperatively.

19. The method as defined in claim 12, further comprising registering the bone element to one of magnetic resonance and ultrasound images of the bone element.

20. A computer assisted surgical system capable of locating and tracking a bone element in three dimensional space, comprising: a bone reference assembly having a trackable member being communicable with at least a sensing element of the system; an anchor member fastenable to the bone element; an articulated support member linking the trackable member to the anchor member; and means for determining a preferred position of the trackable member relative to the sensing element of the system.

21. The system as defined in claim 20, further comprising means for indicating when the trackable member is in the preferred position.

22. The system as defined in claim 21, wherein the means for indicating comprises at least one of a display and an audible signal.

23. The system as defined in claim 20, wherein the articulated support member permits at least two degrees of freedom of the trackable member relative to the anchor member.

24. The system as defined in claim 23, wherein the articulated support member comprises at least two independent joints.

25. The system as defined in claim 24, wherein the joints are rotational joints.

26. The system as defined in claim 25, wherein the joints are lockable.
